(19) United States
(12) Patent Application Publication
(10) Pub. No.: US 2011/0314562 A1
(43) Pub. Date: Dec. 22, 2011

(54) Insect Infection Method for Production of Proteins

(75) Inventors: Mei-Chun Lu, Miaoli County (TW); Chiu-Hsun Liao, Miaoli County (TW); Shih-Chin Yu, Miaoli County (TW)

(73) Assignees: Executive Yuan, Miaoli (TW); Miaoli District Agricultural Research and Extension Station, Council of Agriculture,

(21) Appl. No.: 12/818,825

(22) Filed: Jun. 18, 2010

(51) Int. Cl. C12P 21/00; A01K 29/00

(52) U.S. Cl. 800/4; 119/6.5

(57) ABSTRACT

The present invention provides an insect infection method for use in the production of a protein with a baculovirus expression vector in the insect, the method comprising the steps of:
(g) providing a plurality of insect larvae or pupae;
(h) providing a solution comprising a wild type baculovirus or a baculovirus expression vector having a desired gene encoding a protein;
(i) stressing the insect larvae or pupae;
(j) soaking the insect larvae or pupae in the solution for an appropriate time so that they are infected with the wild type baculovirus or the baculovirus expression vector; and
(k) incubating the infected larvae or pupae for production of the protein; and
(l) harvesting the protein.

The method can treat a great quantity of larvae simultaneously, achieve batch infection of larvae or pupae, save manpower and effectively infect larvae or pupae at a high infection rate.

### FIELD OF THE INVENTION

The present invention relates to an insect infection method for use in the production of a protein with a baculovirus expression vector in the insect. Particularly, the infection method is used to infect insect larvae or pupae.

### BACKGROUND OF THE INVENTION

A low cost alternative to bioreactor-based protein production exists in the use of live insect larvae as “mini bioreactors”. Such an approach uses, in effect, the insect larvae as a factory for manufacture of the desired protein product. Because insect larvae can be grown quickly and inexpensively, there have been attempts to genetically engineer them to express a protein instead of using cells to produce a protein. The gene must be introduced into the larvae to produce the protein. Baculovirus have been used to introduce genes into insects or their larva.

The baculovirus expression vector system is widely used in insects. Baculovirus, an insect virus, was until recently thought to infect only insects and has been utilized as a large-scale expression system of transgenes in insects. Baculoviruses are usually named after the host from which they are isolated. For example, the baculovirus isolated from alfalfa looper was designated Autographa californica (Ac) MNPV. However, baculoviruses which are almost identical to AcMNPV have been found in Trichoplusia ni, Galleria mellonella and Rachiplusia ou. Other baculovirus expression vectors are also known, for example from Bombyx mori (silkworm) (Bm) NPV. Bm NPV vector system is particularly useful for producing recombinant proteins in silkworm larvae, which are easily reared and handled.

The most common methods for infecting insect larvae with virus are oral administration, individual injection and aerosol spray. However, there are several problems which will probably be the bottleneck for practical and industrial utilization of silkworm bioreactor.

U.S. Pat. No. 5,288,616 discloses a process for producing an economic protein by using silkworms, which comprises: orally infecting the silkworms with a virus in which a gene coding the target protein has been inserted; raising the silkworms; and collecting the target protein from the silkworms. EP 0638124 provides pre-occluded baculovirus particles used for oral infection which have been genetically altered so that they lack a functional polyhedrin orgranulin gene. EP 1442658 (and its counterparts: CN1599554; US 2004241822; JP 2003111535; WO 03030637) provides a process for producing a feed for infection with a recombinant virus in silkworms and a method of simultaneously inoculating orally a recombinant virus into silkworms. Toru Arakawa et al. discloses peroral infection of nuclear polyhedrosis virus budded particles in Bombyx mori L., aided by an optical brightener, Tinopal UNPA-GX (Journal of Viorlogical Methods 88 (2000) pp. 145-152). The authors further developed a method of using an insect growth regulator, flufenoxuron, to infect perorally the silkworm Bombyx mori L. with BmNPV (Journal of Virological Methods 100 (2002) pp. 141-147). However, how to increase the oral infectivity of insects with the baculovirus remains a challenging problem in the art. The recombinant baculovirus normally deletes polyhedrin gene for protein production, so these recombinant baculovirus through oral administration will be digested by insects, resulting in a low infection rate. Furthermore, it is difficult to control the dosage when using the oral infection.

The recombinant baculovirus can infect the larvae through individual dorsal injection by a syringe. For example, CN 1974776 uses liposome containing recombinant baculovirus to infect silkworm by injection. Although infection by injection has a higher infection rate; it is time- and labor-consuming JP 9051742 provides an automatic injection device to save manpower. However, it still requires extensive time and cannot treat silkworms in batch. The consumption of time and labor has become a bottleneck for practical and industrial utilization of baculovirus expression system in the silkworm bioreactor.

ROC (Taiwan) Patent No. I255693 provides an aerosol method to infect larva with baculovirus, but it can only be used in third or fourth instars but not fifth instars. U.S. Pat. No. 7,261,886 improves the aerosol infection method to produce recombinant proteins and baculovirus bio-insecticides. However, such aerosol methods cannot infect larva evenly and the infection rate is not satisfactory.

Achieving a high baculovirus infection rate in insects without requiring extensive time and manpower is a challenging problem; there is need for an innovative, infection method that is both efficient and capable of achieving a high infection rate.

### SUMMARY OF THE INVENTION

The present invention provides an insect infection method for use in the production of a protein with a baculovirus expression vector in the insect, the method comprising the steps of:
(a) providing a plurality of insect larvae or pupae;
(b) providing a virus solution comprising a wild type baculovirus or a baculovirus expression vector having a desired gene encoding a protein;
(c) stressing the insect larvae or pupae;
(d) soaking the insect larvae or pupae in the solution for an appropriate time so that they are infected with the wild type baculovirus or the baculovirus expression vector; and
(e) incubating the infected larvae or pupae for the production of the protein; and
(f) harvesting the protein.

The invention also provides a device for practicing the infection method of the invention, comprising a holder for holding a virus solution, a first net located on top of the holder for holding the insect larvae or pupae and a second net with a tenon for adjusting the height of the net.

### BRIEF DESCRIPTION OF THE DRAWINGS

FIG. 1 shows the device for practicing the method of the invention.

FIG. 2 shows Western blot analysis of CSFV E2 accumulation in the body fluid of silkworms after 4 days of inoculation. M: XP protein standard; INF-15: Silkworms infected with INF method performed at 15 minutes; INF-0.5: Silkworms infected with INF method performed at 0.5 hour; INF-1: Silkworms infected with INF method performed at 1 hour; INJ: Silkworms infected with INJ method; Std E2: skE2 186 ng; CK: Health silkworms.

### DETAILED DESCRIPTION OF THE INVENTION

The present invention provides a method of infecting insect larvae or pupae with baculovirus. The method can treat a great quantity of larvae or pupae at a time and achieve batch infection of larvae or pupae. Furthermore, the method of the invention can save time and manpower and evenly and effectively infect larvae or pupae at a high infection rate.

The present invention provides an insect infection method for use in the production of a protein with a baculovirus expression vector in the insect, the method comprising the steps of:
(a) providing a plurality of insect larvae or pupae;
(b) providing a virus solution comprising a wild type baculovirus or a baculovirus expression vector having a desired gene encoding a protein;
(c) stressing the insect larvae or pupae;
(d) soaking the insect larvae or pupae in the solution for an appropriate time so that they are infected with the wild type baculovirus or the baculovirus expression vector; and
(e) incubating the infected larvae or pupae for the production of the protein; and
(f) harvesting the protein.

According to the invention, the term “infection” refers to the development of viral infection, which may include either overt pathology or merely replication and propagation of the virus in an infected animal. In one embodiment, detection of expressed proteins from the chimeric viral genome indicates that the viral construct is infectious.

According to the invention, the term “larva” refers to the immature, wingless, and often wormlike feeding form that hatches from the egg of many insects. It alters chiefly in size while passing through several molts, and is finally transformed into a pupa or chrysalis from which the adult emerges. The term “pupa” refers to a life stage of some insects undergoing transformation. The pupal stage is found only in holometabolous insects, those that undergo a complete metamorphosis, going through four life stages; embryo, larva, pupa and adult. In embodiments of the invention, the insect larva or pupa is that of Bombyx mori (silkworm), Trichoplusia ni Hubner (cabbage looper), Autographa californica (alfalfa looper) or Spodoptera frugiperda. Preferably, the larvae are in the third, fourth or fifth instar.

According to the invention, the “wild-type baculovirus” refers to any the naturally occurring baculovirus. The majority of baculoviruses used as biological control agents are in the genus Nucleopolyhedrovirus. The most common baculovirus is Autographa californica nucleopolyhedrovirus (AcMNPV).

According to the invention, the “baculovirus expression vector” refers to baculoviruses comprising an endogenous or exogenous gene or DNA. The baculovirus expression vector is widely used to express the gene in cultured insect cells and insect larvae. Two of the most common isolates used in foreign gene expression are Autographa californica multiple nuclear polyhedrosis virus (AcMNPV) and Bombyx mori (silkworm) nuclear polyhedrosis virus (BmNPV). According to one embodiment of the invention, the virus is at a concentration of 10⁴-10¹⁰ pfu/ml, preferably, 10⁵-10⁷ pfu/ml.

The gene is preferably either a transgene, which is a gene of interest introduced into target cells, or a marker gene, which is used as an index of isolation or introduction, etc. More preferably, the gene is an exogenous transgene. Furthermore, the gene to be recombined preferably has a promoter, which allows the gene to be expressed at a high level in insect cells. When a final vector adapted to a host is prepared, a promoter which expresses in the cell is used as the transgene promoter. Further, the gene to be introduced is not particularly limited as long as it can be expressed by the above-mentioned promoter; but from the viewpoint of usefulness, genes related to various kinds of genetic diseases, interferon, cytokines, neurotrophic factors, nonself-antigen genes, nucleotide sequences coding virus antigens (such as influenza virus and hepatitis virus), etc., cancer suppressor genes, antisense sequences such as Ras, cancer genes, suicide genes such as thymidine kinase, etc., antibiotic genes, antibody genes or therapeutic protein genes, are preferred. In this connection, the following articles are incorporated herein as reference: Journal of Clinical microbiology, 2009, pp. 3276-3282; Appl Microbiol Biotechnol, 2010, 85:459-470; PLoS ONE, December 2008, Volume 3, Issue 12, e3933, pp. 1-7; and Molecular and Cellular Biology, December 1983, p. 2156-2165.

In one embodiment, the Bombyx mori baculovirus expression vector systems can be used successfully to express endogenous or exogenous genes of interest. Preferably, the exogenous genes can be isolated from a wide range of prokaryotic and eukaryotic organisms and viruses. Representative examples include the expression of a number of human genes, including growth hormone (hGH); macrophage colony-stimulating factor (hM-CSF); beta-interferon (HuIFN-beta); human alpha-interferon in Bombyx mori larvae and CD4 (T cell surface protein T4) replaced by the signal DNA sequence from the insect signal peptides coding for the cuticle gene or adipokinetic hormone. The silkworm larvae has also been used for high-level expression and secretion of biologically active mouse interleukin-3 and recombinant ookinete surface antigens of Plasmodium berghei. A recombinant, full length keratinocyte growth factor (KGF) has been expressed in insect cell hosts including Bombyx mori. (U.S. Pat. Nos. 5,863,767; 5,843,883 and 5,773,586). A prokaryotic prolylendopeptidase from Flavobacterium has been expressed in insect cells using Bombyx mori nuclear polyhedrosis virus (BmNPV) (U.S. Pat. No. 5,521,081).

Components from viruses also can be expressed in the Bombyx mori baculovirus expression vector system. These include classical swine fever virus; porcine circovirus, swine influenza virus, pseudorabies virus, porcine coronavirus, foot-and-mouth disease virus, the fusion glycoprotein (F) from Newcastle disease virus (NDV) strain D26; the kinase-active v-erbB gene, an oncogene of the avian erythroblastosis virus encoding a protein that is a truncated version of the epidermal growth factor receptor; hepatitis B and C virus antigens; characterization of v-sis protein; recombinant proteins from human T-cell leukemia virus type I (HTLV-I); and human papillomavirus type 6b CSFV E2 gene product with DNA-binding activity in insect (Bombyx mori) cells.

According to the invention, the term “stressing” refers to the larvae or pupae being put under emotional or physical pressure, without resulting in death. After being stressed, the larvae or pupae will recover and express proteins normally. The invention unexpectedly found that the stressing of larvae or pupae in combination with the soaking of larvae or pupae provide improvements in infection. In embodiments of the invention, the stressing can be achieved by the following means, alone or in combination: keeping larvae or pupae at a low temperature or a temperature higher than the normal growth temperature but lower than the tolerance temperature, starving larvae or pupae, placing larvae or pupae in an environment with reduced atmosphere, irradiating larvae or pupae with radiation or treating larvae or pupae with a chemical agent. In one embodiment, the larvae or pupae are kept at a low temperature from about 2° C. to about 15° C., preferably, about 3° C. to about 15° C., more preferably, about 4° C. to about 15° C., about 4° C. to about 12° C., about 5° C. to about 15° C., or about 4° C. to about 10° C., most preferably, about 4° C. to about 6° C., about 4° C. to about 8° C. or about 4° C. to about 10° C. In another embodiment, the larvae or pupae are kept at a higher temperature of about 30° C. to 45° C., preferably, about 32° C. to 45° C., about 32° C. to 42° C., about 32° C. to 40° C., about 35° C. to 45° C. or about 35° C. to 40° C. In a further embodiment, the larvae or pupae are treated by irradiating with a low dose of UV. In another further embodiment, the larvae or pupae are treated by being placed in an environment with a reduced atmosphere. More preferably, the atmosphere is reduced 5% to 50%. In another further embodiment, the larvae or pupae are fasted for at least two days. Preferably, the larvae or pupae are fasted for two, three or four days.

According to the invention, the “soaking” of insect larvae or pupae is performed by soaking the insect larvae or pupae in the baculovirus solution so that they are infected with the baculovirus without dying. The virus will enter the stomas of the larvae or pupa for infection. Preferably, the soaking time of the larvae or pupae is at least 5 minutes. Preferably, the soaking time ranges from 5 minutes to 6 hours, more preferably, 10 minutes to 6 hours, 15 minutes to 1 hour, 30 minutes to 1 hour, 30 minutes to 2 hours or 30 minutes to 3 hours. Persons having ordinary knowledge in the art can adjust soaking time and virus concentration on the basis of species, physiological condition, species of heterologous gene, etc.

According to the invention, the larvae or pupae are incubated for the production of protein. Preferably, the incubation time is 1.5 to 5 days, 1.5 to 6 days, 1.2 to 4 days, 1.5 to 3 days or 2 to 3 days. Effective incubation conditions and duration varies according to species of larva or pupa, incubation temperature and proteins, as is known in the art. For example, ideal growth temperature for silkworms is about 25° C. to about 28° C.

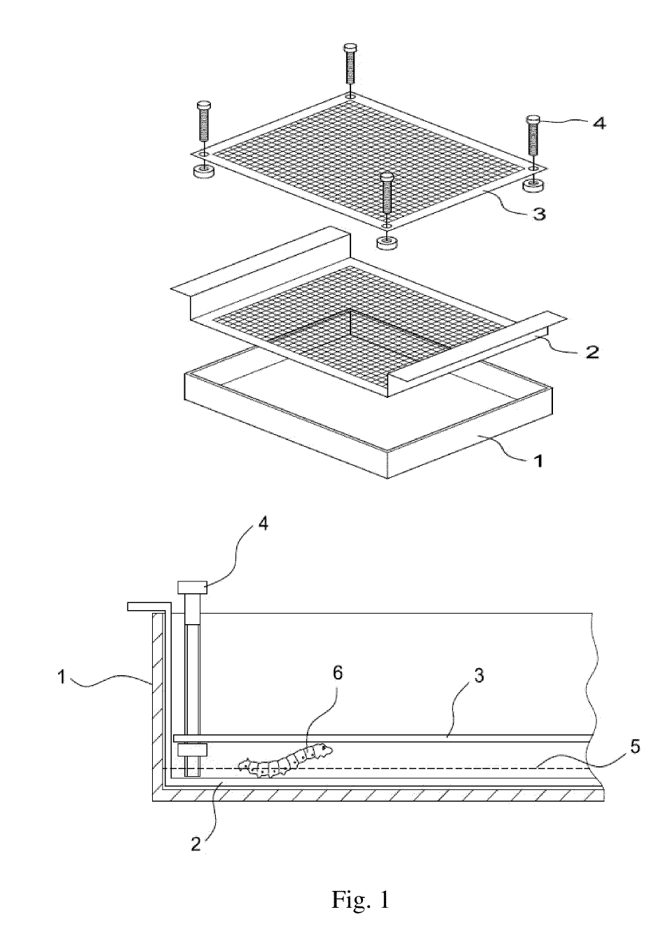

The present invention also provides a device used to practice the infection method of the invention, comprising a holder for holding virus solution, a first net located on top of the holder for holding the insect larvae or pupae and a second net with a tenon for adjusting height of the net. Preferably, the device further comprises a vacuum means to reduce the concentration of atmosphere. The representative figure of the device of the invention is shown in FIG. 1. The device comprises a holder 1 for holding virus solution 5. The first net 2 is located on top of the holder for holding the insect larvae or pupae and the second net 3 with a tenon 4 for adjusting height of the net so that the larvae 6 or pupae can be contained between the first net and the second net and they can be adequately soaked by the virus solution. Preferably, the net is a stainless steel or plastic net. The device of the invention can treat a number of larvae or pupae in batch. The dimensions of the device can be adjusted depending on the amount of larvae or pupae.

The infection method of the invention provides a high infection rate; preferably higher than 85%, more preferably, higher than 90%. The infection method of the invention does not require extensive manpower and can infect numerous larvae or pupae simultaneously.

### EXAMPLE

### Example 1

### Infections of Silkworm Larvae with Bombyx mori Baculovirus (BmNPV) and Infection Rate Assay

The fifth instar silkworm larvae Bombyx mori OJ03*OJ04 at the first day were inoculated with recombinant Bombyx mori baculovirus (BmNPV) with red fluorescent protein by aerosol infection, injection infection, oral infection and the infection method of the invention. The uninfected silkworms served as negative control.

For aerosol infection, 2 ml of the recombinant BmNPV solution at a concentration of 1×10⁷ pfu/ml was sprayed several times onto silkworm larvae and mulberry leaves. Injection infection was conducted by injecting BmNPV solution with 1×10⁶ pfu/ml to stomas of the silkworm larvae by microinjection. As for feeding injection, mulberry leaves coated with the recombinant BmNPV solution with 1×10⁷ pfu/ml were fed to the silkworm larvae.

For the infection method of the invention, the silkworm larvae were placed at 4° C. for 15 hours. After 1 hour, larvae were soaked in BmNPV solution with 1×10⁷ pfu/ml for 1 hour.

The resulting silkworms were incubated at 25+/−2° C. with high humidity. After they were inoculated for 4 days, the infection rates (larvae expressing transformed protein/infected larvae) of various infection methods were determined, as shown in Table 1 below:

TABLE 1

| Infection Method | Infection Rate (%) |
|---|---|
| Injection | 95 +/− 5^{ab} |
| Method of the Invention | 93.3 +/− 5.8^{b} |
| Aerosol Spray | 0 |
| Oral Infection | 0 |
| Negative Control | 0 |

^{b}less than 95% significant difference;
^{ab}equal to or more than 95% significant difference.

As shown in the table, the infection method of the invention achieves an infection rate as high as that of the injection method while requiring less labor allowing the ability to treat a large number of silkworms in mass.

### Example 2

### Expression Level of Red Fluorescent Protein in Infected Silkworms

Twenty-five (25) silkworm larvae of Bombyx mori species, OJ03*OJ04, were used in this study. The BmNPVs were constructed so that they contained a nucleic acid sequence of red fluorescent protein (RFP). The nucleic acid sequence of rfp is known in the art (Geoffrey, S. B., D. A. Zacharias, and R. Y. Tsien. 2000. Biochemistry, mutagenesis, and oligomerization of DsRed, a red fluorescent protein from coral. PNAS 24:11984-11989.). The construction of the recombinant BmNPV was conducted according to the process mentioned in Maeda, S. 1989. Expression of foreign genes in insects using baculovirus vectors. Ann. Rev. Entomol. 34:351-372.

The injection infection (INJ) and the infection method of the invention (INF) were used to infect the silkworms with the above-mentioned recombinant BmNPV. The infections and inoculation of the silkworms were performed and the infection rates were determined according to Example 1. The body fluids of the silkworms were collected to determine the expression level of RFP by fluorescence spectrophotometer (Zenyth 3100, Bio-Rad Co.). The results are shown in Table 2 below:

TABLE 2

| Infection | Infection Rate (%) | RFP Level (μg/μl) |
|---|---|---|
| INJ | 86.3 ± 12.5 | 3.3 ± 1.4 |
| INF | 96.7 ± 3 | 3.6 ± 1.6 |

As shown in the table, the infection method of the invention has infection rates and expression levels as high as those of the injection method.

### Example 3

### Western Blot Assay

The BmNPVs were constructed so that they contained classical swine fever virus (CSFV) E2 antigen. The nucleic acid sequence of CSFV E2 antigen and the construction of the recombinant Bm NPV are known in the art.

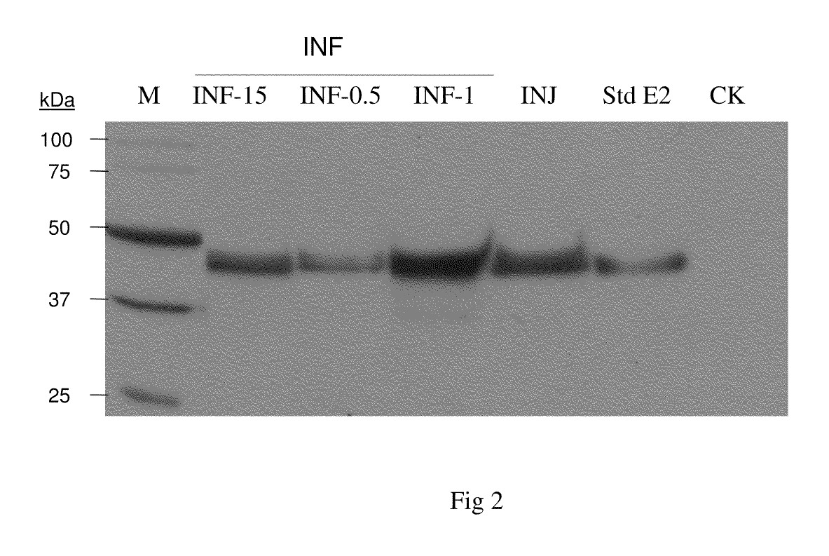

The injection infection (INJ) and the infection method of the invention (INF) were used to infect the silkworms with the above-mentioned recombinant Bm NPV. The health silkworms were used as control. The INF method of the invention was performed at different infection times (15 minutes, 0.5 hour and 1 hour). The resulting silkworms were incubated at 25+/−2° C. with high humidity. After they were inoculated and incubated for 4 days, body fluids of the silkworms were collected for subsequent analysis. The vaccine proteins contained in the body fluid were analyzed by SDS-PAGE electrophoresis (Sambrook and Russell, 2001, Molecular Cloning, A8.40-A8.55). Western Blot immunoassay using PVDF membrane was further conducted. FIG. 2 shows CSFV E2 accumulation in the body fluid of silkworms after 4 days of incubation and suggests that the method of the invention can infect silkworms effectively.

What is claimed is:

1. An insect infection method for use in the production of a protein with a baculovirus expression vector in the insect, the method comprising the steps of:
(a) providing a plurality of insect larvae or pupae;
(b) providing a virus solution comprising a wild type baculovirus or a baculovirus expression vector having a desired gene encoding a protein;
(c) stressing the insect larvae or pupae;
(d) soaking the insect larvae or pupae in the solution for an appropriate time so that they are infected with the wild type baculovirus or the baculovirus expression vector; and
(e) incubating the infected larvae or pupae for production of the protein; and
(f) harvesting the protein.

2. The infection method of claim 1, wherein the insect is Bombyx mori (silkworm), Trichoplusia ni Hubner, Autographa californica (alfalfa looper) or Spodoptera frugiperda.

3. The infection method of claim 1, wherein the insect is Bombyx mori (silkworm) or Trichoplusia ni Hubner.

4. The infection method of claim 1, wherein the larvae are in the third, fourth or fifth instar.

5. The infection method of claim 1, wherein the baculovirus is Autographa californica multiple nuclear polyhedrosis virus (AcMNPV) or Bombyx mori (silkworm) nuclear polyhedrosis virus (BmNPV).

6. The infection method of claim 1, wherein the gene is an endogenous or exogenous transgene.

7. The infection method of claim 1, wherein the gene relates to vaccine protein, antibacterial protein, various kinds of genetic diseases, interferon, cytokines, neurotrophic factors, nonself-antigen genes, nucleotide sequences coding virus antigens (such as influenza virus and hepatitis virus), etc., cancer suppressor genes, antisense sequences such as Ras, cancer genes, suicide genes such as thymidine kinase, etc., antibiotic genes, antibody genes or therapeutic protein genes.

8. The infection method of claim 1, wherein the stressing in step (c) is performed by the following means, alone or in combination: keeping larvae or pupae at a low temperature or a temperature higher than the normal growth temperature but lower than the tolerance temperature, starving larvae or pupae, placing larvae or pupae under an environment with reduced atmosphere, irradiating larvae or pupae with radiation or treating larvae or pupae with a chemical agent.

9. The infection method of claim 1, wherein the stressing in step (c) is performed by keeping the larvae or pupae at a low temperature of about 2° C. to about 15° C.

10. The infection method of claim 9, wherein the temperature is about 3° C. to about 15° C., more preferably, about 4° C. to about 15° C., about 4° C. to about 12° C., about 5° C. to about 15° C., or about 4° C. to about 10° C., most preferably, about 4° C. to about 6° C., about 4° C. to about 8° C. or about 4° C. to about 10° C.

11. The infection method of claim 9, wherein the temperature is about 4° C.

12. The infection method of claim 1, wherein the stressing in step (c) is performed by keeping the larvae or pupae at a high temperature of about 30° C. to 45° C.

13. The infection method of claim 12, wherein the stressing in step (c) is performed by keeping the larvae or pupae at a high temperature of about 32° C. to 45° C., about 32° C. to 42° C., about 32° C. to 40° C., about 35° C. to 45° C. or about 35° C. to 40° C.

14. The infection method of claim 1, wherein the stressing in step (c) is performed by irradiating the larvae or pupae with a low dose of UV.

15. The infection method of claim 1, wherein the soaking time of the larvae or pupae is at least 5 minutes.

16. The infection method of claim 1, wherein the soaking time of the larvae or pupae ranges from 5 minutes to 6 hours.

17. The infection method of claim 1, wherein the soaking time of the larvae or pupae ranges from 10 minutes to 6 hours, 15 minutes to 1 hour, 30 minutes to 1 hour, 30 minutes to 2 hours or 30 minutes to 3 hours.

18. The infection method of claim 1, wherein the stressing in step (c) is to treat larvae or pupae by placing them in an environment with a reduced amount of atmosphere.

19. The infection method of claim 18, wherein the atmosphere is reduced by 5% to 50%.

20. The infection method of claim 1, wherein the stressing in step (c) is performed by fasting the larvae or pupae for at least 2 days.

21. The infection method of claim 1, wherein the stressing in step (c) is performed by fasting the larvae or pupae for two, three or four days.

22. The infection method of claim 1, wherein the virus is at a concentration of 10-10¹⁰ pfu/ml.

23. The infection method of claim 1, wherein the virus is at a concentration of 10⁵-10⁷ pfu/ml.

24. A device for practicing the infection method of claim 1, comprising a holder for holding a virus solution, a first net located on top of the holder for holding the insect larvae or pupae and a second net with a tenon for adjusting the height of the net.

25. The device of claim 24, wherein the first or second net is a stainless steel or plastic net.
